(19) United States
(12) Patent Application Publication
(10) Pub. No.: US 2005/0256539 A1
(43) Pub. Date: Nov. 17, 2005

(54) Methods and systems for using transcranial magnetic stimulation to enhance cognitive performance

(76) Inventors: Mark S. George, Sullivan's Island, SC (US); Daryl E. Bohning, Warren, CT (US)

Correspondence Address: NEEDLE & ROSENBERG, P.C., SUITE 1000, 999 PEACHTREE STREET, ATLANTA, GA, 30309-3915, US

(21) Appl. No.: 10/509,262

(22) PCT Filed: Mar. 25, 2003
(86) PCT No.: PCT/US03/09463

§ 371 Date: May 11, 2005

Related U.S. Application Data
(60) Provisional application No. 60/367,520, filed on Mar. 25, 2002.

(51) Int. Cl. A61N 1/00

(52) U.S. Cl. 607/2; 600/13

(57) ABSTRACT

Methods and systems are provided for using transcranial magnetic stimulation (TMS) to enhance cognitive performance of at least one subject. At least one neural circuit is located in the brain of the subject, which is activated when the subject performs a predetermined task. Functional magnetic resonance imaging maps may be used to scan and generate maps of the interested neural circuits so as to locate proper neural circuits responsible for a predetermined task. An electromagnetic coil is positioned over a region on the scalp of the subject corresponding to the at least one neural circuit in the brain of the subject. A transcranial magnetic stimulation is delivered from the coil to the region on the scalp of the subject to induce current to flow in the brain that causes neuronal depolarization in the brain and effectuates a change in the performance of the predetermined task by the subject.

### BACKGROUND

The present invention generally relates to the use of transcranial magnetic stimulation to enhance performance. More particularly, the present invention relates to methods and systems for using transcranial magnetic stimulation to enhance cognitive performance of one or more subjects.

For over a century, it has been recognized that electricity and magnetism are interdependent (Maxwell's equations) (Bohning, 2000). Passing current through a coil of wire generates a magnetic field perpendicular to the current flow in the coil. If a conducting medium, such as the brain, is adjacent to the magnetic field, current will be induced in the conducting medium. The flow of the induced current will be parallel, but opposite in direction, to the current in the coil (Cohen et al., 1990; Brasil-Neto et al., 1992; Saypol et al., 1991; Roth et al., 1991). Thus, transcranial magnetic stimulation (hereinafter “TMS”) has been referred to as “electrode-less” electrical stimulation to emphasize that the magnetic field acts as the medium between electricity in the coil and induced electrical currents in the brain.

TMS involves placing an electromagnetic coil on the scalp. Subjects are awake and alert. There is some discomfort, in proportion to the muscles that are under the coil and to the intensity and frequency of stimulation. Subjects usually notice no adverse effects except for occasional mild headache and discomfort at the site of the stimulation.

High intensity current is rapidly turned on and off in the coil through the discharge of capacitors. This produces a time-varying magnetic field that lasts for about 100-200 microseconds. The magnetic field typically has a strength of about 2 Tesla (or 40,000 times the earth's magnetic field, or about the same intensity as the static magnetic field used in clinical MRI). The proximity of the brain to the time-varying magnetic field results in current flow in neural tissue.

The technological advances made in the last 15 years have led to the development of magnetic stimulators that produce sufficient current in brain to result in neuronal depolarization. Neuronal depolarization can also be produced by electrical stimulation, with electrodes placed on the scalp (referred to as transcranial electric stimulation (“TES”)). Importantly, unlike electrical stimulation, where the skull acts as a massive resistor, magnetic fields are not deflected or attenuated by intervening tissue. This means that TMS can be more focal than TES. Furthermore, for electrical stimulation to achieve sufficient current density in brain to result in neuronal depolarization, pain receptors in the scalp must be stimulated (Saypol et al., 1991).

A striking effect of TMS occurs when one places the coil on the scalp over the primary motor cortex. A single TMS pulse of sufficient intensity causes involuntary movement. The magnetic field intensity needed to produce motor movement varies considerably across individuals and is known as the motor threshold (Kozel et al., 2000; Pridmore et al., 1998). Placing the coil over different areas of the motor cortex causes contralateral movement in different distal muscles, corresponding to the well-known homunculus. TMS can be used to map the representation of body parts in the motor cortex on an individual basis. Subjectively, this stimulation feels much like a tendon reflex movement. Thus, a TMS pulse produces a powerful but brief magnetic field which passes through the skin, soft tissue and skull and induces electrical current in neurons, causing depolarization which then has behavioral effects (body movement).

Single TMS over the motor cortex can produce simple movements. Over the primary visual cortex, TMS can produce the perception of flashes of light or phosphenes (Amassian et al., 1995). To date, these are the ‘positive’ behavioral effects of single pulse TMS. Other immediate behavioral effects are generally disruptive. Interference with, and perhaps augmentation of, information processing and behavior is especially likely when TMS pulses are delivered rapidly and repetitively. Repeated rhythmic TMS is called repetitive TMS (rTMS). If the stimulation occurs faster than once per second (1 Hz) it is modified as fast rTMS.

rTMS at frequencies of around 1 Hz has been shown to produce inhibition of the motor cortex. rTMS at higher frequencies of several minutes has been shown to excite the underlying cortex for several minutes. Manipulations of frequency and intensity may produce distinct patterns of facilitation (fast rTMS) and inhibition (slow rTMS) of motor responses with distinct time courses. These effects may last beyond the duration of the rTMS trains with enduring effects on spontaneous neuronal firing rates. Determining whether, in fact, lasting increases and decreases in cortical excitability can be produced as a function of rTMS parameters, and whether such effects can be obtained in areas outside of the motor cortex, are of key importance.

As indicated above, TMS is generally safe with no side effects except mild headache in about 5% of subjects. However, higher frequency TMS can produce seizures. With the publication of safety tables in 1998, there have been no unintended seizures produced in the world (Wassermann et al., 1996b; Wassermann, 1997; Wassermann et al., 1996a). Animal studies, along with human post-mortem and brain imaging studies (Nahas et al., 2000a), have all failed to find any pathological effects of TMS (Lorberbaum & Wassermann, 2000).

TMS evoked motor responses result from direct excitation of corticospinal neurons at or close to the axon hillock. It is thought that the TMS magnetic field induces an electrical current in superficial cortex. The TMS magnetic field declines exponentially with distance from the coil. This limits the area of depolarization with current technology to a depth of about 2-cm below the brain's surface. Nerve fibers that are parallel to the TMS coil (perpendicular to the magnetic field) are more likely to depolarize than those perpendicular to the coil. It is thought as well that bending nerve fibers are more susceptible to TMS effects than straight fibers (Amassian et al., 1995).

Conventional TMS coils are either round, or in the shape of a figure eight (Cohen et al., 1990). The figure eight designs are more focal than the round coils. Most coils are mere copper wire either alone or wrapped around a solid metal core. Because most coils are inefficient, they produce heat as a byproduct. The solid coils are more efficient, without a heating problem. Other manufacturers have used water cooling (Cadwell) or air cooling (Magstim) to deal with this issue. DARPA materials science research might drastically improve the current technology.

The peak effect of TMS can be localized to within less than a millimeter in terms of functional location. More work is needed in terms of actually understanding the exact location of TMS effects (Bohning et al., 2001; Bohning et al., 1997). There is much debate about whether one could devise an array of coils in such a way as to stimulate deep in the brain without overwhelming superficial cortex.

Although current TMS technology has shown that it can interrupt and facilitate many behaviors, several technical issues limit the field. Current coils are bulky and hard to focus. Because of the materials used, they overheat and require large capacitors. It is not clear whether one could stimulate deeper in the brain with current designs. Finally, TMS at present is limited to single TMS applications. There has been virtually no work done on producing arrays of TMS coils that are discharged in a coordinated fashion. Such an array would likely vastly open up new TMS vistas (including defense applications).

Most studies with TMS have shown that high frequency TMS can interrupt a higher cognitive function. It is relatively easy to produce speech arrest by stimulating over the motor speech area (Broca's). Speech arrest occurs only for the moment of stimulation. With the MUSC team as consultants, Drs. Stern and Lisanby at Columbia are using this ‘knockout lesion’ ability of TMS to understand the neural circuits used in response to sleep deprivation. There are also some studies using precisely timed single pulse TMS for augmentation of function. But as with the other potential applications, this requires precise timing (Grafinan, 2000). Precisely timing TMS bursts with stimulus presentation would thus be more difficult to adapt to warfare conditions.

At the recent CAPS teaming workshop, two different Armed Forces Representatives highlighted the need for ‘non-pharmacological’ approaches to boosting cognitive performance. There is thus likely only a small psychological hurdle for general warfighter acceptance of these techniques, should we succeed in finding ways of using TMS to enhance performance at baseline or following sleep deprivation. This general warfighter acceptance is remarkable given how revolutionary these concepts are, compared with the status quo. These representatives, when asked why there might be such easy acceptance of TMS in the battlefield, responded that TMS made sense in terms of focal delivery of the needed changes, without distribution throughout the whole body (e.g. lack of side effects), and the ability to turn the device on and off without worry about lingering half-lives.

Thus, there is a huge need for radical new approaches (as highlighted in the CAPS announcement). Human limitations on performance are now the rate limiting aspects of most weapons systems and warfare capability. There is thus also likely easy acceptance of TMS, if the necessary background work shows that it can improve performance. The potential impact on DOD could thus be huge. If TMS is found to boost normal performance, or even slightly restore performance in the face of sleep deprivation, there will undoubtedly be many other potential military and non-military applications. The design of man-portable TMS systems would provide the foundation for a revolution in the field of TMS, with profound impact on therapeutic applications as well.

Therefore, there is a need to develop new methods and systems that can utilize TMS to deliver stimulation to proper neural circuits of a living being to enhance cognitive performance of the living being.

### SUMMARY

According to a first aspect of the invention, methods for using TMS to enhance cognitive performance are provided. According to a first embodiment, a method for enhancing cognitive performance includes the steps of locating at least one neural circuit in the brain of a subject, which is activated when the subject performs a predetermined task, positioning an electromagnetic coil over a region on the scalp of the subject corresponding to the at least one neural circuit in the brain of the subject, delivering a transcranial magnetic stimulation from the coil to the region on the scalp of the subject, inducing a current to flow in the brain, causing neuronal depolarization in the brain, and effectuating a change in the performance of the predetermined task by the subject.

According to another embodiment, a method of using TMS to enhance cognitive performance in a plurality of subjects, such as human beings, includes the steps of dividing the plurality of subjects into groups, subjecting each of the groups into a first state and a second state, locating at least one neural circuit in the brain of a subject in the group corresponding to one of the first state and the second state, which is activated when the subject performs a predetermined task under one of the first state and the second state, positioning an electromagnetic coil over a region on the scalp of the subject corresponding to the at least one neural circuit in the brain of the subject, delivering a transcranial magnetic stimulation from the coil to the region on the scalp of the subject, inducing a current to flow in the brain, causing neuronal depolarization in the brain, and effectuating a change in the performance of the predetermined task by the subject under one of the first state and the second state. In one embodiment, the first state is a state at which a subject is at rest, and the second state is a state at which a subject is sleep-deprived. fMRI can be utilized to identify different neural circuits associated with different subject at a state, wherein the neural circuits are activated while a predetermined task is performed. TMS can then be delivered to proper neutral circuits to restore and/or retrain the circuits to enhance the performance.

According to yet another embodiment, a method of using TMS to enhance cognitive performance in at least one subject includes the steps of during a behavior individualized imaging of at least one cognitive neural circuit, locating the at least one cognitive neural circuit, individually positioning an electromagnetic coil over a region on the scalp of the subject corresponding to the at least one cognitive neural circuit, and delivering a stimulation through the electromagnetic coil to the at least one cognitive neural circuit to affect the behavior related to the at least one cognitive neural circuit. Individualized imaging can be performed by an fMRI scanner, and the electromagnetic coil is associated with a TMS system, which are interleaved to provide synergistic stimulation(s).

In another aspect, a system of using TMS to enhance cognitive performance is provided. In one embodiment, the system includes means for locating at least one neural circuit in the brain of a subject, which is activated when the subject performs a predetermined task, an electromagnetic coil that can be positioned over a spot on the scalp of the subject corresponding to the at least one neural circuit in the brain of the subject, means for delivering a transcranial magnetic stimulation from the coil to the spot on the scalp of the subject so as to induce a current to flow in the brain, cause neuronal depolarization in the brain, and effectuate a change in the performance of the predetermined task by the subject. The locating means includes an fMRI system that can be utilized to scan and generate maps of the interested neural circuits so as to locate proper neural circuits responsible for a predetermined task. Additionally, the system includes a computer having a CPU and one or more memory devices to, among other things, coordinate the operation among the different parts of the system, optimize the operation parameters such as TMS use parameters, and facilitate the TMS delivering.

In another embodiment, a portable system of using TMS to enhance cognitive performance in at least one subject includes an energy source, such as a battery, a CPU, a database having fMRI maps of neural circuits corresponding to a plurality of tasks stored therein, and a movable electromagnetic coil. The CPU is electrically coupled to the energy source and communicates with the database, which is associated with a memory device of the CPU, or alternatively with a separate memory device, or both. The movable electromagnetic coil is electrically coupled to the energy source and communicates with the CPU. In operation, when a subject is to perform a predetermined task, the CPU communicates with the database and selects one or more fMRI maps of one or more neural circuits corresponding to the predetermined task. The CPU then communicates with the movable electromagnetic coil so that the electromagnetic coil to be positioned over a region on the scalp of the subject according to the selected one or more fMRI maps. Proper transcranial magnetic stimulation from the coil is then delivered to the region on the scalp of the subject so as to induce a current to flow in the brain, cause neuronal depolarization in the brain, and effectuate a change in the performance of the predetermined task by the subject. The subject can be a person or an animal. The system can be constructed within a frame that is portable. Alternatively, the system can have an array of TMS coils, each being able to deliver TMS individually or in coordination.

These and other aspects will become apparent from the following description of the preferred embodiment taken in conjunction with the following drawings, although variations and modifications may be effected without departing from the spirit and scope of the novel concepts of the disclosure.

### BRIEF DESCRIPTION OF THE DRAWINGS

FIG. 1 is a flow chart schematically showing one method of using TMS to improve cognitive performance according to an exemplary embodiment.

FIG. 2 shows an exemplary transverse structural scan of a subject.

FIG. 3 illustrates how a TMS is placed over a subject according to an exemplary embodiment.

FIG. 4 shows exemplary reaction times and error rates for one subject.

FIG. 5 illustrates a TMS in cooperation with a fMRI scan according to an exemplary embodiment.

FIGS. 6 and 7 schematize and scale exemplary relationships between the magnetic field of a TMS coil and induced currents v. brain activation, respectively.

FIG. 8 illustrates shows exemplary results relating to the relative positions of TMS-induced thumb movement and a similar movement executed volitionally.

FIG. 9 illustrates an exemplary image of the brain with the fMRI activation in the motor cortex superimposed.

FIG. 10 illustrates how to use MRI to image the magnetic field of TMS according to an exemplary embodiment.

FIG. 11 shows the magnetic field of a TMS coil on surfaces at different depths according to exemplary embodiments.

### DETAILED DESCRIPTION

Several exemplary embodiments of the invention are now described in detail. Referring to the drawings, like numbers indicate like parts throughout the views. As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise.

Overview

Recent researches suggest the potential of TMS technologies. Topper et al. have shown that stimulation over temporal lobe facilitates or improves picture naming (Topper et al., 1998). Grafman et al. have recently shown that stimulation over the prefrontal cortex, and not sham stimulation, improves analogous reasoning (Boroojerdi et al., 2001). The same NIH group has shown that 1 Hz TMS for 10 minutes can transiently suppress motor cortex or visual cortex activity, for up to 20 minutes following stimulation.

However, there has never been a systematic, large-scale attempt to understand these phenomena and hence to use the TMS technologies in real world applications. This invention represents exciting improvements in the field and provides methods and system for ways of delivering TMS to improve performance. Several aspects, applications, and embodiments of the present invention are reviewed as follows.

In one application, prior to engaging in a task, a subject such as a soldier has TMS applied to the appropriate region, with improved cognition for a short amount of time so as to improve the subject's performance in the task. A slight modification would be to have intermittent stimulation while performing the task at low intensities that does not interfere with cognition, which in fact improves reasoning.

According to exemplary embodiments, this and other applications can be realized through:
1. Improvements in TMS technology as discussed below; and
2. A series of TMS studies and/or experiments performed according to exemplary embodiments in healthy adults providing novel methods, procedure(s) and discoveries:
   a. A series of excitatory TMS over prefrontal or other regions of the brain to improve cognitive reasoning for a period of time at a first state of mind such as being awake;
   b. Whether this improvement is measurable on tests resembling real word conditions such as combat conditions and whether it degrades accuracy;
   c. Finding and optimizing use parameters for achieving this and other effects;
   d. Locating other regions where intermittent stimulation might improve cognitive reasoning; and
   e. Repeat all of the above in subjects who are in the second state of mind such as sleep deprived.

In other words, according to exemplary embodiments, TMS and fMRI may be used to understand the neural circuits involved and to apply TMS at proper regions in order to produce these effects (Nahas et al, 2000b; George & Bohning, 2000; Bohning et al, 2000a; Bohning et al, 2000b; and Bohning et al, 1998).

Thus, the present invention greatly advances the TMS field, with spin-offs in the use of TMS as a neuroscience tool, in the ability to use TMS in other cognitive applications, and in advancing TMS as a potential therapy for neuropsychiatric disorders. Some of these spin-offs include:
Materials science—New types of TMS coils are made from new materials that have different electrical conductivity and may be made to stimulate deeper into brain than presently possible;
Size of TMS devices—New types of TMS coils are made in ways that are smaller, without the inefficiencies that generate heat;
Arrays—A plurality of TMS coils are coordinated in an array to stimulate multiple regions in precisely timed ways. Arrays may be received in a helmet for portability. Such a helmet may have many overlapping figure eight regions that are controlled from an external device such as a central control center; and
Portability—In one embodiment of the present invention, the stimulators; capacitors and energy source are able to be placed in combat areas in a portable device without sacrificing other functions.

Furthermore, among other things, the present invention demonstrates, using one representative task (such as the Sternberg), whether and how TMS at different use parameters can be used to improve performance. Interestingly, even in studies over the motor cortex, there has not been a synthetic systematic examination of TMS use parameters on a behavior.

In sum, the present invention has the potential to revolutionize this very promising young field of TMS applications.

In one aspect, the invention relates to a method of determining TMS Use Parameters. The measures here are performance data while healthy subjects are performing the Sternberg (Study 1A1) and having TMS delivered over candidate or control regions, at different TMS use parameters. Specifically, response time (milliseconds) and error rates are measured. This will be done both at rest and with partial sleep deprivation (4 hours of sleep) (Study 1A4).

A separate set of data contains the fMRI brain maps of regions activated during the Stemberg at baseline. These fMRI brain maps can be used to guide the TMS placement, as well as to determine the physical dimensions of a man-portable TMS system that, for example, do not need to have each soldier perform an fMRI activation map at the field.

Another set of data contains the fMRI brain maps of regions activated during the Stemberg while TMS is being applied within the scanner (1A5). These maps may help in understanding how TMS is modifying the brain during task performance, and to generate hypotheses about secondary sites where TMS might be applied in order to even further boost performance.

Generation alone of these three data sets (behavioral dose response with focused, image guided application; data on the variation across subjects in the physical location of this function; data on how TMS applied at a key site modifies brain activity) represents a remarkable and never before completed advancement for science.

In another aspect, the invention relates to provide several man-portable TMS coil systems. In one embodiment of the present invention, this section of work provides three models of potential TMS systems. Using high power computers, each design is tested using computer modeling for basic design issues such as (weight, heat generation, internal stresses, the amount of induced electrical current it could deliver within the brain), and the power needed to run such a system. This results in a series of design drawings of several TMS options, and formal white paper discussions of each area under testing (coil, power systems, etc). The designs can be optimized by optimizing the use parameters needed to improve cognition—(e.g. frequency, intensity, total dose, temporal relationship of TMS to task performance), as well as information derived from the model testing.

Among other things, the present invention has several advantages over the prior art as discussed below. Many of the prior art studies focused on pharmacological agents to improve performance, with little focus on where in the brain the compound is acting to improve performance. (It was interesting to note the general level of acceptance from military spokesman about the preference for non-pharmacological (i.e. TMS) CAP boosts over pharmacological approaches). On the other hand, many groups proposed using functional brain imaging (PET or fMRI) to understand CAP variables, but without discrete methods of acting on this circuit or systems level knowledge. This invention is unique in that it directly translates systems level circuit findings from functional brain imaging, and then uses this knowledge to apply TMS to improve performance.

There are others who are proposing to use TMS in other applications. Importantly, the other proposals lack the design and testing elements of how to translate TMS into real world such as war fighter applications. Their approaches also focus on understanding sleep deprivation effects, while this invention has improved baseline or near baseline functioning.

Thus, the present invention is unique and highly relevant to the CAPS mission. One aspect of the present invention is to determine if TMS can boost performance on a sleep-deprivation sensitive task at baseline (or under minimal degradation of performance), and then build a system that may allow this to be translated to the field.

In one embodiment of the present invention, fMRI image is used to guide placement of the TMS coil so that the TMS coil is located at an important region for the behavior. This maximizes the efficiency of finding performance enhancing TMS use parameters. Another aspect of the present invention involves examining the fMRI activation maps and determining probabilistic rules for TMS application, which may solve how one can translate these initial image guided effects with a simpler to use formula for large-scale production.

In sum, the present invention provides a revolutionary new approach to boosting cognitive performance, TMS, both at baseline and then potentially during periods of sleep deprivation. It uses high-tech functional brain imaging and frameless stereotaxy to explore TMS use parameters over fMRI identified critical regions, and then uses a very unique technology, interleaved TMS within an fMRI scanner, to fully explore potential ways of using TMS to boost performance.

Methods and System for Determining TMS Use Parameters

According to an exemplary embodiment, a series of TMS studies or experiments may be conducted with the goal of determining whether TMS can enhance cognitive performance. The task that is used for these studies is the Sternberg task. This is chosen because it is easily performed, with a large body of literature, is stable over time and largely immune from learning and order effects, and is sensitive to changes in sleep, with measurable decreases in task performance following sleep deprivation. Alternatively, other tasks can be chosen and/or used.

Determining TMS use parameters involves a series of TMS studies in the BSL and the CAIR 3T fMRI scanner, specially built for interleaved TMS/fMRI studies. The studies flow logically from one to the next, and are linked one on the next, although only 1A3 is truly conditional on prior results. They involve a common task, and a common method of delivering TMS to a particular region. The different studies are labeled as follows:
1A1—TMS during Sternberg; 30 subjects
1A2—TMS preceding Sternberg; 30 subjects
1A3—Replication and refinement of TMS effects; 60 subjects (conditional)
1A4—Testing TMS effects with minor sleep deprivation; 60 subjects
1A5—Using interleaved TMS/fMRI to understand TMS effects and find synergistic stimulation (20 subjects)
2A6—Using previously acquired fMRI scans to determine the probabilistic method of applying TMS in a man-portable system
2A7—Testing whether probabilistic TMS placement is as effective as fMRI guided
2A8—Testing whether similar effects are found in women.
   Note that the number of subjects for each study can be modified to include less or more subjects.

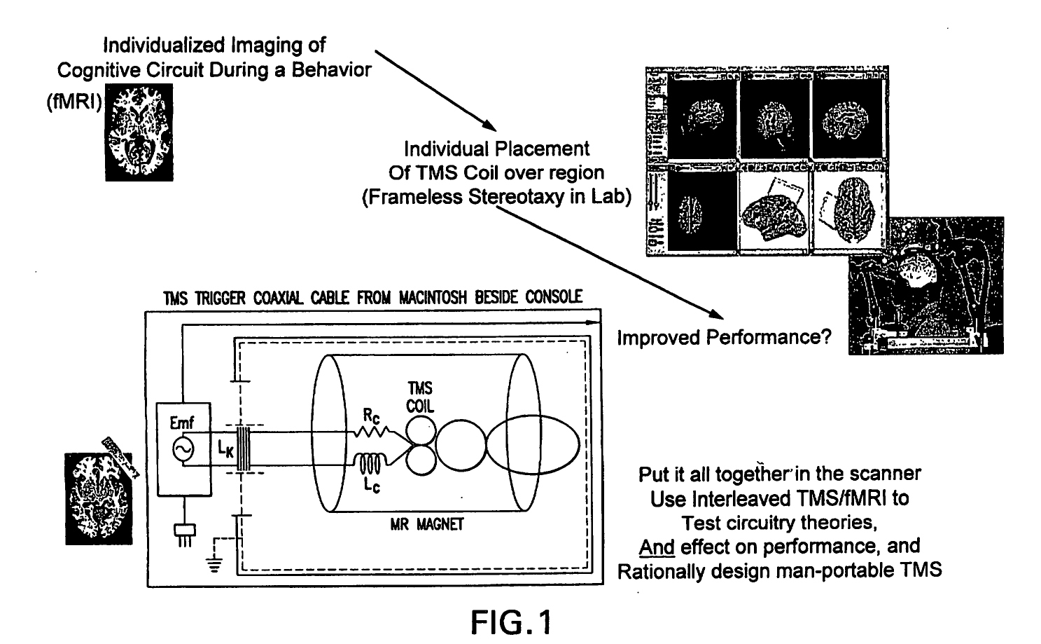

These studies are performed according to the embodiments of the present invention. Most of these studies involve methods recently worked out according to the present invention as illustrated in FIG. 1, which is discussed in detail below.

fMRI Scan. Within a 1.5 Tesla Philips MRI scanner, subjects were given a high-resolution structural MRI scan, followed by an echoplanar BOLD fMRI scan. During the fMRI scan, subjects had their heads constrained and were able to view stimuli through MRI compatible 3-D goggles. They were also able to respond to stimuli using a two-button response pad. Subjects were then shown the Sternberg task, or a control task including seeing objects and responding to their physical location. The task and control stimuli were presented in block designs of 26 seconds each, alternating over 10 minutes. Subjects were trained on the Sternberg task prior to MRI scanning.

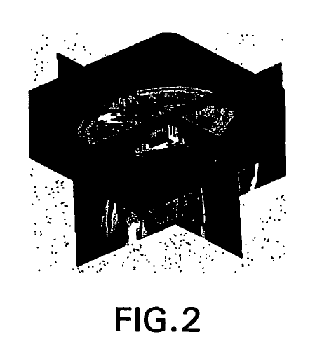

Image Data Analysis. Images were then transferred from the Philips to a computer system such as the MUSC MAIAL Sun systems. There they were inspected for artifacts, and corrected for motion across the 10 minutes. They were spatially and temporally smoothed using SPM within the software MEDx. Staying within the person's own brain space (that is, not transforming the data to a common brain space), t-tests were performed on the data to discern brain regions that were significantly more active during the Sternberg task than during the control condition. Regions that were significant at the p<0.001 level were then subjected to a cluster analysis of p<0.05. These functional difference maps were then overlaid on the same person's structural MRI scan. The MUSC MAIAL has the ability to perform this series of steps in less than 24 hours. FIG. 2 shows a transverse structural scan of a subject. Also shown are the brain regions that are significantly more active while performing the Sternberg compared to the control task (p<0.001 for display).

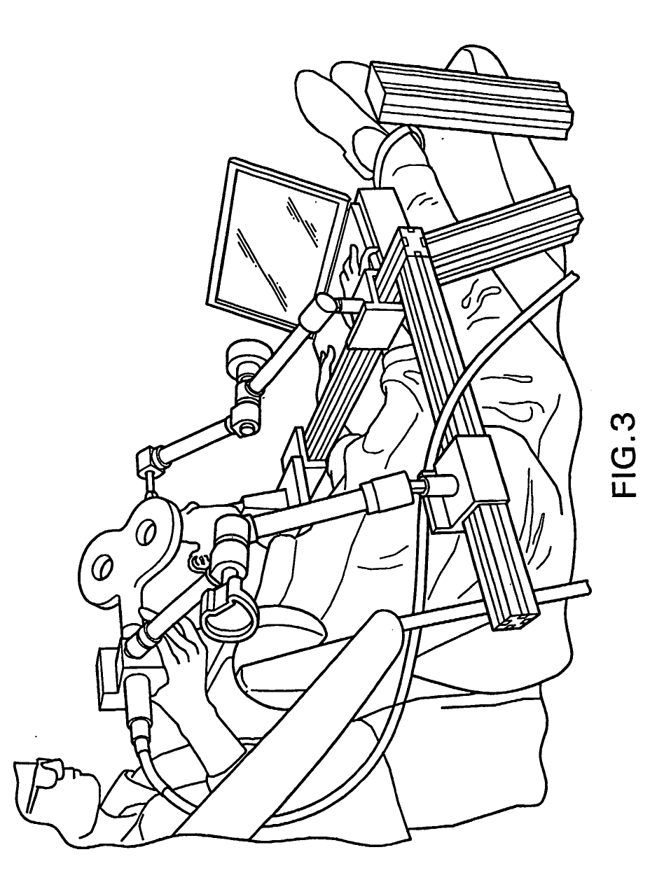

Frameless Stereotaxy. Within the BSL—The MUSC BSL is a beta test site for a frameless stereotaxy system such as one developed at McGill University (Brainsight). These merged structure/function Sternberg images are then transferred to the BSL Brainsight system. The subject is then placed in a modified dental chair with passive head immobilization system. The subject's brain is then stereotactically linked to the fMRI image. The TMS coil is then placed over the subject's brain region, overlying the prefrontal area of maximal activation during the Sternberg minus control conditions as shown in FIG. 3. The TMS coil is also positioned over a region in the secondary occipital cortex not activated in the fMRI images.

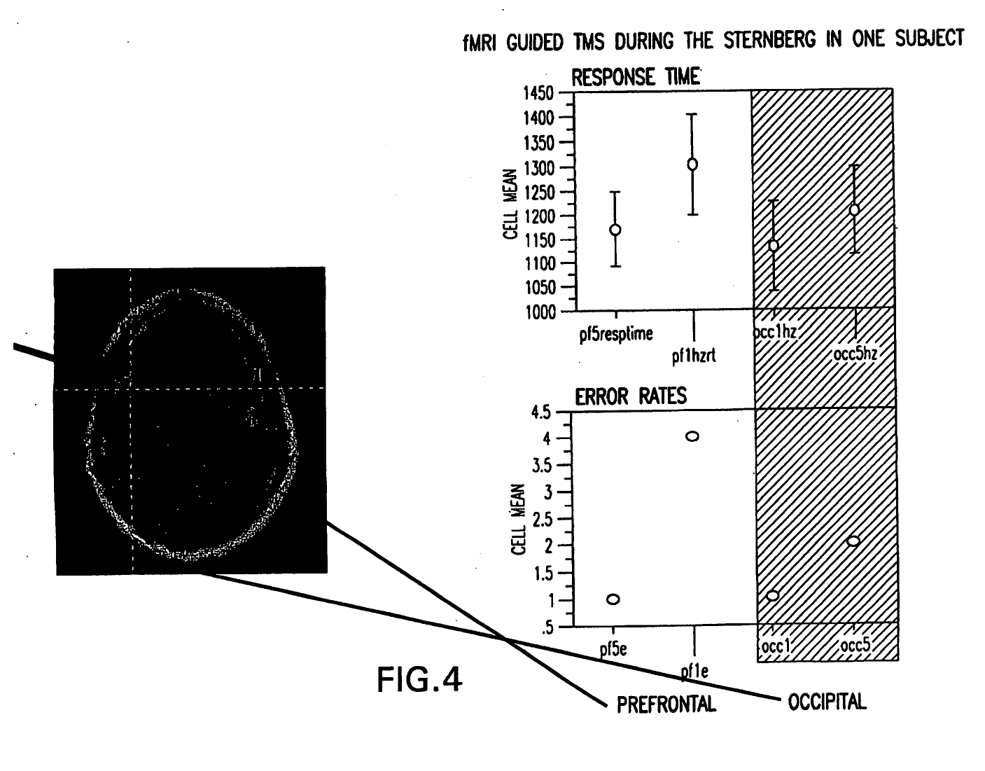

Sternberg Testing with TMS using different use parameters. Then, with the TMS coil positioned over the candidate region or control region, in a randomized, counterbalanced manner, the subject performs the Sternberg with TMS applied at different use parameters. FIG. 4 shows the reaction times and error rates for one subject who was stimulated over the prefrontal or occipital regions, each at high frequency (5 Hz) or low frequency (1 Hz), all at 110% of MT.

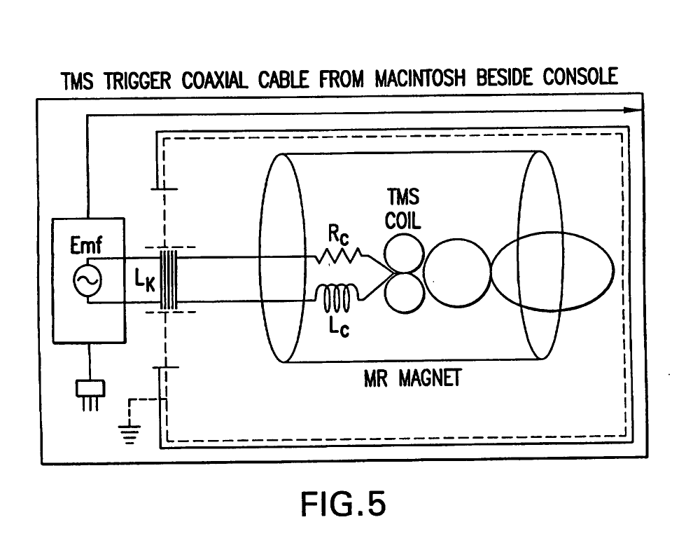

Follow-up Testing using the Interleaved TMS/fMRI technique. Once a TMS use parameter is found that has a significant positive effect, then subjects can be placed within the fMRI scanner as shown in FIG. 5, and will perform the Sternberg task, with and without TMS applied to the region. This allows an understanding of how TMS is acting to improve behavior, and it may also identify secondary sites where TMS might be applied with synergistic effects.

Applicants have spent several years reasoning through the most efficient methods for rationally determining how to apply TMS in cognitive paradigms. The method described herein shows the approach that the applicants determined as the most logical, and the most efficient, at determining how to use TMS to modify cognition and improve performance. This individual fMRI based method of TMS placement, although technically complex, completely solves the issue of where to apply the TMS. With this method, there is no question that the TMS is being delivered at the appropriate location. One can then begin a rational dose finding exploration. When specific dose or use parameter effects are found, the same images that were used to guide the TMS placement in individuals can be examined for probabilistic rules about where to apply TMS short of within individual fMRI guidance.

Several studies or experiments performed according to exemplary embodiments involving TMS delivered over fMRI identified regions to test if there are frequencies that enhance performance are now described in more detail.

Subjects: For all experiments in this aspect of the present invention, the applicants recruit healthy young men (age 18-35), with the following inclusion and exclusion criteria: No history of major head trauma or seizures; Medically healthy; No brain diseases; No metal objects in their bodies prohibiting an MRI scan; No history of Substance Abuse; Urine drug screen negative.

Subjects are studied while free of alcohol or coffee for the day, in a non-sleep deprived state.

Also, several studies presented here are not involving female subjects. This has several advantages. First, there may be gender differences in regional brain activity and response to TMS. We would have to immediately double our sample sizes and all dosing work. Further, brain excitability and response to TMS changes slightly over the menstrual cycle, and we would need to perform studies in women timed in conjunction with the menstrual cycle. However, the methods and systems described herein can be applied to female subjects.

Subjects are invited to come to the BSL for initial screening. There they may give written informed consent, undergo a history and physical examination, undergo minor initial cognitive testing, be trained on the Sternberg, and may provide a urine sample for drug screen.

On the second visit they may then have an fMRI brain scan (3.0 Tesla, Philips MRI scanner). This gives a high quality structural scan for Brainsight registration, and gives circuit information about Sternberg task performance. This information can be processed in near real-time (24 hours) in the MAIAL, using MEDx and SPM. This generates an activation map within each individual, merged onto that person's brain.

Two days later they return to the BSL where they will then participate in the following TMS techniques. Motor threshold is determined using standard techniques. In the following, several experiments are described:

Study 1A1. 30 subjects, each studied twice TMS During the Stemberg. In a complex, 6-hour study, subjects have the Magstim figure eight coil placed over the prefrontal regions identified on MRI scanning. TMS is delivered in the following matrix, with the following 3 variables tested (intensity, frequency, and region): The intensities tested will be—90, 110, 120% of MT. The frequencies tested are 1, 5, 10 Hz. Other frequency values can also be chosen. All epochs last about 10 minutes, with a 10-minute rest. This gives 3×3×2=18 combinations of these variables. 18×20 minutes=360 minutes (or 6 hours). Studies normally start at 8 am and proceed for 2, 3-hour blocks, with a 30-minute lunch break in between. A standard lunch is provided. Each subject then returns for another similar day, separated by at least one-day rest. The order of variables are randomized and counterbalanced. Performing the same studies twice on each individual can help reduce noise and standard deviation and greatly enhance the ability to find TMS induced cognitive improvement, if it exists.

TMS is delivered regardless of whether the Sternberg stimulus is being displayed, the interstimulus interval is on, or a response is needed. The reasoning for this is that if TMS needs to be that precisely coupled to stimulus processing, it is unlikely to have field applications.

Reaction Time and Error Rates: Data obtained for each subject averaged across the two days by condition (e.g. profrontal 110% MT 5 Hz results from each day will be summed and averaged within each subject). These mean RT and error rates are analyzed using a 3 factor ANOVA testing for location (prefrontal or occipital control), frequency (1, 5, 10), or intensity (90%, 110%, 120% MT). Post-hoc tests are utilized to explore behavioral trends within each factor.

Sample Sizes and Power: 30 subjects, each with two TMS day-long sessions, provide ample power to detect improvement in Sternberg performance. Note that if positive effects are found, these are subject to later replication, so this sample size is designed to be able to identify an effect, if it exists. This can be tested later for replication. Sample sizes of 20-30 have been used in the literature to show TMS effects on cognition of 10% or more. A formal power analysis reveals that if there is a 5% variation of Stemberg performance across the variables, a sample of 30 subjects will have an Alpha of 0.05 and a 95% power to detect a 5% improvement as a function of one of the 3 factors (intensity, site, frequency). Further, for all effects found, these are tested for replication in a separate cohort (See Study1A3 below).

Study 1A2: 30 subjects are split in 2 groups. TMS before Sternberg is being performed. Data from the Queen Square group over motor cortex and from Cohen and colleagues over both motor and occipital cortex have shown that TMS can be delivered before a cognitive task, with resulting lasting effects for up to an hour. In many ways, delivering TMS before a behavior in a real world setting such as a combat setting is advantageous to performing it during the behavior.

In a similar, 6 hour study, 15 initial subjects have the Magstim figure eight coil placed over the prefrontal site or the occipital cortex. TMS are delivered in the following matrix, with the following 3 variables tested (intensity, frequency, region): In this study, TMS is delivered during the 10 minutes immediately preceding the Stemberg task. There will be no TMS during the actual task. Rather, subjects are examined for lasting effects of TMS delivered before the task. The intensities tested are—90, 110, 120% of MT. The frequencies tested are 1, 5, 10 Hz. All epochs will last 10 minutes, with a 10 minute rest. This gives 3×3×2=18 combinations of these variables. 18×20 minutes=360 minutes (or 6 hours). Studies normally start at 8 am and proceed for 2, 3-hour blocks, with a 30-minute lunch break in between. Each subject then returns for another similar day, separated by at least one day off. The order of variables are randomized and counterbalanced. Performing the same studies twice on each individual helps reduce noise and standard deviation and greatly enhances the ability to find TMS induced cognitive improvement. Again, other sets of testing parameters can be utilized to provide new set of data.

Results are analyzed in the first 15 subjects using the 3 factor ANOVA described above. These variables can be examined and predict the use parameters in the next 15 subjects. In this study, the same methods as above are used, except the frequency and intensity with the best effect are utilized, and a new variable of time preceding task is introduced. Thus there is one frequency and one intensity. TMS will be delivered for variable amounts of time (10 min, 20 min, 30 min) and variable times before the Sternberg (10 min, 20 min, 30 min).

Again, these response data (reaction time, error rate) are analyzed using repeated measures ANOVA examining for dose and time from TMS.

Study 1A3: Study 1A3 is designed to, among other things, examine replication and refinement of TMS effects. If significant effects are found with either Study IA1 (During) or 1A2 (Preceding) or both, an attempt is made to replicate these in an independent cohort and examine TMS effects with parameters in the same neighborhood. Thus, these conditional studies in 30 subjects are designed to replicate and extend any findings that might be observed in the initial studies. To do so, an additional 30 subjects are recruited and TMS are performed either during or before the Sternberg so as to explore the neighboring use parameters. For example, if prefrontal TMS 1 Hz, MT were found to enhance the Sternberg during performance, one would retest this, and add the conditions of 1.5 Hz, 0.5 Hz, 90% MT and 110% MT.

These studies (1A1, 1A2, 1A3) are designed to be performed in subjects who are not sleep deprived. One can question, however, whether effects found in these subjects are able to be applied to the CAP mission of improving performance during sleep deprivation, which is for each subject a different state from the state where a subject is not sleep deprived. It is possible, although unlikely, that a TMS effect seen in non-sleep deprived conditions might have a paradoxical effect in sleep deprived conditions. More likely is another scenario where TMS is able to have little or a marginal effect under optimum conditions, but a greater effect in slightly degraded conditions. The following two studies are similar to the above studies, but differ in that subjects are admitted overnight to a test facility such as the GCRC and then awakened at 3 am, thus ensuring that they are partially sleep deprived during the TMS session the following day.

Study 1A4 (during and before Sternberg, with partial sleep deprivation): 60 subjects. The same studies as described above are done with subjects who are partially sleep-deprived. That is, subjects are admitted to the GCRC and awakened at 3 am, and then have testing done on the day following sleep deprivation. They will be sent home for a normal night's sleep, then readmitted the following night and retested, thus having a second day of repeated mild partial sleep deprivation. These studies are needed even if large performance enhancing TMS effects are seen in the earlier, non-sleep deprived studies. These subjects would be run in this study for safety and to make sure that TMS stimulation parameters that are helpful in non sleep-deprivation are not problematic or worsening in sleep deprived individuals.

Study 1A5: 20 subjects. Study 1A5 utilizes TMS within the fMRI scanner to examine effects found. When significant effects are found in any of the Studies 1A1, 1A2 and 1A3, as they do, TMS is then performed within the fMRI scanner at the location with use parameters found to have a behavioral effect. This study provides valuable information about how TMS is producing its effects, and might indicate other regions where TMS could be applied in a synergistic fashion in addition to prefrontal stimulation. This study may also aid greatly in understanding how focal or diffuse the TMS application might be in order to achieve behavioral effects.

In doing so, TMS is administered within the fMRI scanner at the use parameters found in the earlier studies, while subjects are performing the Sternberg task. Data analysis is performed similar to that used to determine the optimum spot for TMS placement.

Study 2A6: To answer how precise must the placement be to achieve enhancement in performance, Study 2A6 is designed to perform image data analysis. Thus, in this study, no new subjects, but examination of imaging data previously acquired. If performance effects are found using the very precise anatomical precision method, there is a need to see if TMS can be done in a less precise anatomic location method. The functional imaging scans obtained in the earlier parts of the Studies 1A1-1A5 will be analyzed in MedX to generate a probabilistic rule for how imprecise to place the TMS coil and still improve performance. This may involve examining the range of regions identified as critical in Sternberg performance across subjects and then morphing individual brain scans into a common brain space.

Study 2A7: Among other things, study 2A7 is designed to test whether probabilistic TMS placement is as effective as fMRI guided. In this study, the rule generated in Study 1A6 is directly tested in a new cohort of 30 subjects. The use parameters are those previously identified as optimum for maximizing effects. The variable to be explored here is location of the TMS coil—fMRI guidance, versus the rule-based algorithm identified above. In addition to a direct test of the proposed algorithm, TMS is systematically delivered in 1-mm increments away from the MRI identified region, to assess how imprecise the TMS application might be.

Study 2A8: Among other things, Study 2A8 is designed to test whether similar effects are found in women. In one embodiment, the test group includes 30 women. In particular, before a TMS system could be used in the military, it would be important to determine if similar effects are seen in women. Thus, in 30 women screened as above, they would have TMS applied in the manner determine in Study 1A1-1A5 testing to have optimum effects.

An Experiment:

In yet another experiment/study, a plurality of subjects was divided into two groups. The first group contained 6 subjects with clear improvements in the Stemberg (RT) with the prefrontal TMS. The second group contained 6 subjects with clear worsening. Each group then was monitored for each subject's activity during the Stemberg within the fMRI, wherein no TMS was applied. Data was obtained and then analyzed to show that the TMS prefrontal improvers (the first group) used prefrontal cortex and cingulated during the Stemberg; in contrast, those in the second group whom TMS did not improve RT, or even worsened it, did not use prefrontal and cingulated but instead used parietal and visual cortex (and/or maybe basal ganglia). Thus, different people use different neural circuits, both at rest and when sleep deprived, and these circuits can be identified and selected by fMRI, which in turn allows TMS to be used to either restore function or train or refrain circuits according to the present invention.

B. Designing A Portable TMS System

Another aspect of the present invention relates to a portable TMS system that can be used in real world situations to enhance cognitive performance. To achieve effective TMS with a man-portable system, there are 3 major considerations: 1) physical size of the TMS “coil”, 2) device positioning, and 3) power requirements. With all three, there are conflicting demands on the system. A small sized “coil” would be more portable, but, if too small, might not be effective at stimulating the brain. A system that would allow the coil's position to range over an area would make it possible to accommodate the varied brain anatomy of different people, but would likely be more bulky and complex. Increased power demands almost always introduce complexity and weight, but decreasing the power of the system would also likely mean that greater accuracy would be required in coil positioning.

Improved understanding of the absolute requirements for effective TMS gained in practicing the present invention provides clearly defined constraints for the design, and innovations in materials and/or design, though not eliminating these conflicts, may reduce them so that a practical man-portable TMS system is achievable according to the present invention.

A portable TMS system according to an exemplary embodiment, takes into consideration at least some of the following:
1) TMS anatomical characterization, i.e., where in the cerebral cortex does neuronal discharge occur: a) sulcus, b) crown of gyrus, c) transition zone, or d) a combination of them;
2) TMS physical characterization, i.e. functional relation between TMS “coil” field and degree of neuronal activation;
3) TMS “coil” coverage requirements, i.e. a) localized or nonlocalized stimulations, b) single or multiple stimulation sites, c) field profile requirements, full widths at half maximum (FWHM): Wx,Wy,Wz;
4) TMS “coil” positioning requirements, i.e. must the coil position be adjustable to accommodate different brain anatomy, range of movement: Rφ, Rθ, Rr;
5) Magnetic field characteristics of concept coils: standard figure-8, modified figure-8, programmable lattice, spinning magnet, moving magnet, and phased-array;
6) Conductivity model of human brain;
7) Electric field characteristics of concept coils: standard figure-8, modified figure-8, programmable lattice, spinning magnet, moving magnet, and phased-array;
8) Induced current characteristics of concept coils: standard figure-8, modified figure-8, programmable lattice, vibrating permanent magnet, and phased-array;
9) TMS induced current measurement project: To determine if TMS induced currents might be mapped in-vivo;
10) Power requirements for TMS stimulator;
11) Preliminary design for reduced power TMS stimulator; and
12) Concepts for man-portable power supply.

Some aspects of design characteristics of a portable TMS system according to an exemplary embodiment are discussed in more detail below.

Magnetic Field and Induced Currents Versus Brain Activation

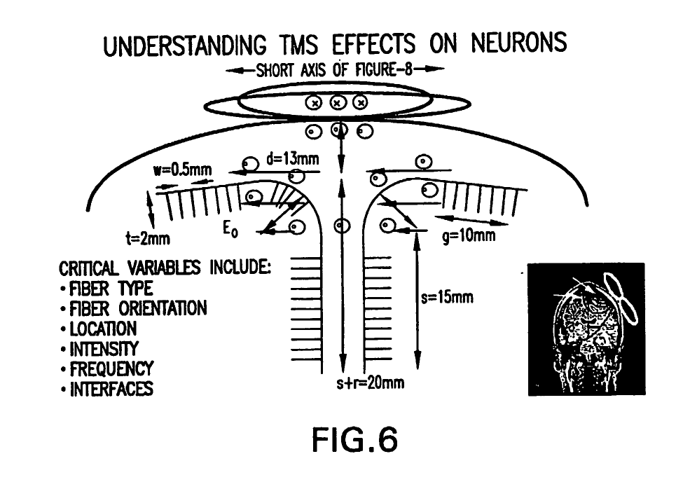

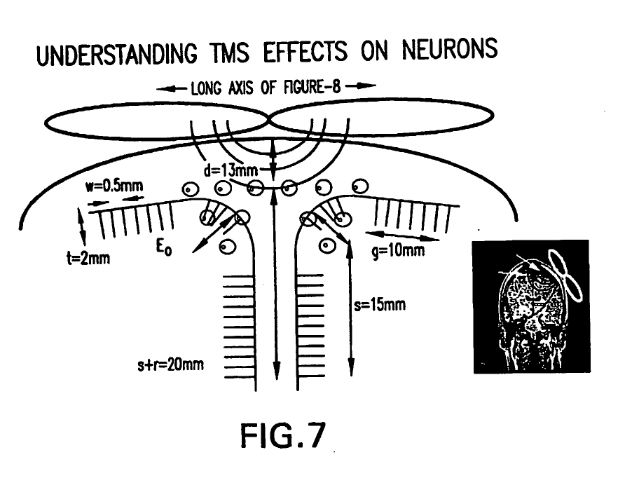

Better data is needed on the relationship between the magnetic field of the TMS coil and the induced currents, and, in turn, the anatomy of cerebral cortex and the actual depolarization of neurons in cerebral cortex. FIGS. 6 and 7 schematize and scale these relationships for orthogonal orientations of a standard figure-8 TMS coil to help more quantitatively define some of the factors involved in TMS, factors which will likely constrain the design of a man-portable TMS system. This provides information about where one needs to stimulate, how focal the stimulations needs to be, and the preferred direction of the induced electric fields. It gives information on the variation in brain anatomy between individuals. Combining this information, one can get an idea of the required size of the “coil” and if it will be necessary to position it differently for different brain anatomies, i.e., if one size fits all, or if one needs to custom fit the “coil” to each person. It also provides information about the field intensities that must be produced, and how they must be directed relative to the relevant structures in the brain. This is important for deciding on a configuration for the coil as well as the power requirements.

Data about size and location of TMS Performance related activations and coil position relative to activation are also needed. Data may come from BrainSight data from Studies 1A1-1A5. This information tells one about the importance of position, i.e., is the effect sensitive to position, or can a generalized stimulation be used.

Data about spatial variation of relevant brain structures are obtained to tell one whether a single configuration is sufficient, or, at the other extreme, if the coil must be customized for or adaptable to each individual. This largely involves a comprehensive literature review of published data (human and animal) and then examination of the Brainsight data being collected in Study 1A2-1A5 subjects.

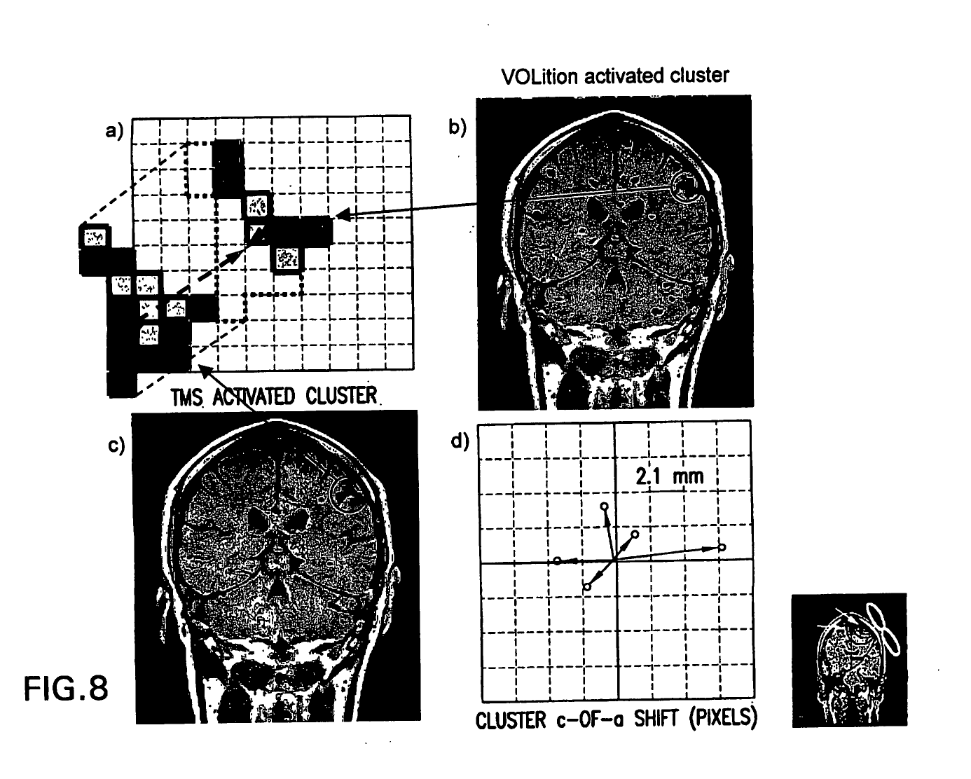

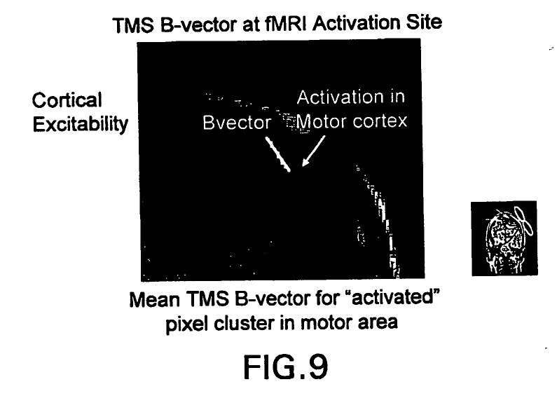

Data about magnetic field vector at activation site can be obtained from two ways. First, phase maps and TMS/fMRI data from past studies are utilized: Using data from previous TMS/fMRI studies over motor cortex, the relative displacements of TMS coil, motor cortex, and fMRI activation will be obtained. These will then be combined with simulations of the TMS coils magnetic field and knowledge of the anatomy of motor cortex to gain a better understanding of the relationship between magnetic field and cerebral cortex for optimum stimulations. FIG. 8 shows exemplary results according to the present invention. The relative positions of TMS-induced thumb movement and a similar movement executed volitionally are determined (Bohning et al., 2000). Coil and magnetic field distributions may be added to complete the picture relating activation and magnetic field. FIG. 9 shows more of this preliminary work on this problem, an image of the brain with the fMRI activation in motor cortex superimposed and an arrow indicating the directions and magnitude of the magnetic field at the center of the fMRI activation.

Additionally, the computed B-field of the coil combined with BrainSight Data from Studies 1A1-1A5 can be utilized. As in the above case, this can provide data about the relative displacements of the TMS coil stimulation and the area of activation in motor cortex. Though it may not show the area of activation as does fMRI activation, it can show the position of the TMS coil position at which the maximum motor evoked potentials (MEPs) were induced relative to brain anatomy. This facilitates one to tie magnetic field to area of cerebral cortex in which neuronal discharge occurs as well as the anatomical structure.

Magnetic Field, Electric Field and Induced Current Simulations for Different Coil Designs

The field patterns of the different coil configurations are a major factor in either eliminating a particular coil design or selecting it for further development. In one embodiment, the coil is capable of stimulating the desired area(s) effectively, and if focal rather than diffuse stimulation is desired, it must not stimulate other areas.

Field Simulations are performed for standard figure-8, modified figure-8, programmable lattice, vibrating “crescent” permanent magnet, and phased-array. The simulations will be similar, except that the electric field induction is caused by the movement by the vibrating permanent magnets, rather than by current pulses as in the figure-8 and lattice coil. Alternatively, field simulations can be conducted on computer models.

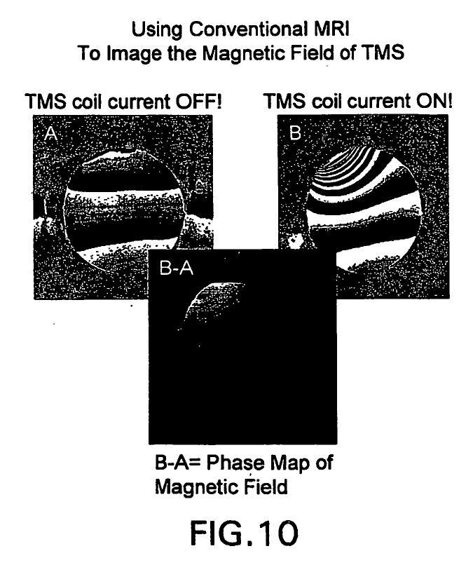

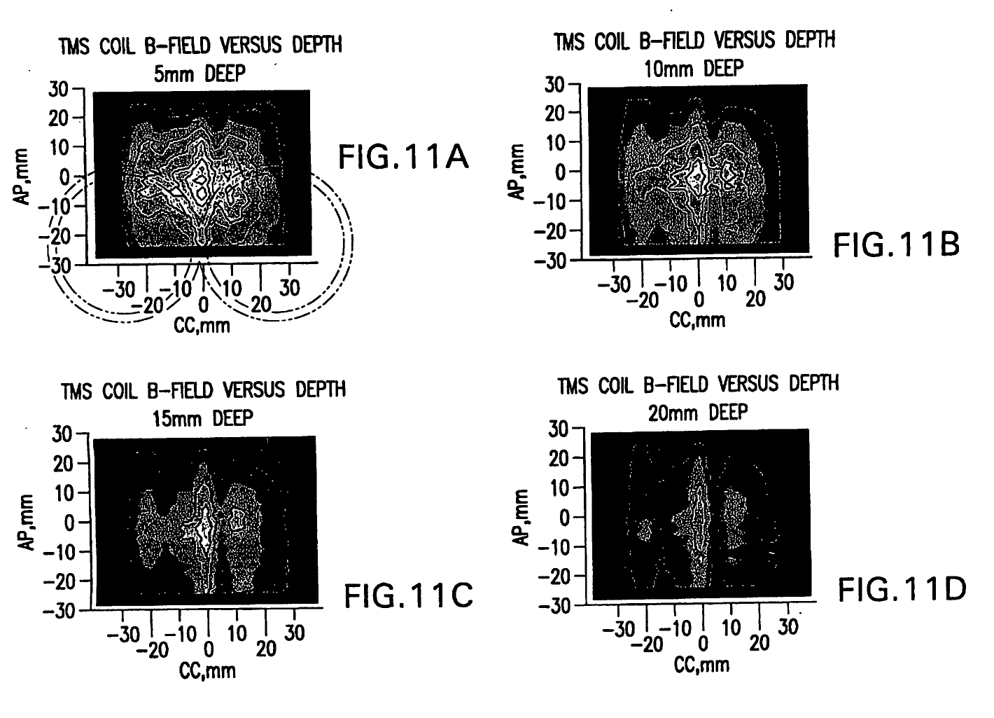

B-field phase maps are utilized as well. The coils magnetic field pattern can be measured using MRI phase maps to check the computed magnetic fields (Bohning et al., 1997). FIG. 10 illustrates the principle, and FIG. 11 shows brain images on which a surface approximately 2 cm below the scalp, about the depth of most TMS and at different depths.

MRI segmentation into gray and white matter and CSF for conductivity volume map are used in the present invention. Moreover, phase map fMRI technique is used to advance understanding in this area.

Induced E-Field Simulations:

The induced E-field may be computed in a plane parallel to a simple figure-8 coil and homogeneous medium. It would be necessary to do this for a volume encompassing the areas of the brain to be stimulated and extend the calculations to, at least, a three component model of brain tissue, i.e., gray and white matter and CSF.

Induced J-Currents Simulations:

Induced currents actually cause the neuronal discharge associated with the activation.

Induced E-Field and Induced J-Currents Versus Brain Anatomy Mapping:

It has been estimated that the transmembrane current flow needed to depolarize the membrane is caused by the spatial derivative of the electric field along the axon, dE/dx, (Reilly 1992; Abdeen and Stuchly 1994; Garnham et al. 1995) and that the peak spatial derivative needed to achieve stimulation is approximately 5 kV/m² (Rudiak and Marg 1994). Our simulations along with previously acquired data and data from the above studies should make it possible for us to check and extend these observations.

Power Requirements:

Based on the strength-duration relation for neuronal depolarization,

$$\begin{matrix}Q ( t ) = {Q}_{0} [ 1 + \frac{Δ t}{Δ {t}_{0}} ] \\ J ( t ) = {J}_{0} [ 1 + \frac{Δ t}{Δ {t}_{0}} ]\end{matrix} \}$$

where Q₀=minimum threshold depolarization charge density for nonleaky membranes
J₀=rheobase, the minimum stimulus current density that can attain threshold at infinite duration
Δt₀=the strength-duration time constant (chronaxie) (≈150 μs, Barker et al., 1991) and our estimates of induced currents, we will attempt to adjust the TMS pulse waveform to increase depolarization efficiency and reduce power consumption.
   Moving Magnet Induction

Though there are no resistive losses due to large currents, the “moving” magnets have to be moved in approximately a quarter of a millisecond. This will require some sort of electromechanical device, which itself will consume power, to overcome the coil's inertia, move it a short distance and then return, which could be cumbersome and fragile. A magnet spinning at high speed, would require neither a large current, nor the generation of the forward and backward impulses of a pulsed system, but would require power to bring the spinning magnet up to speed and keep it there and would generate current continuously not pulses. There would also be a problem with the gyroscopic effect of any magnet, large enough to stimulate the brain, spinning at high speed. In addition, there is no data on the stimulation pattern of such a TMS “coil”, so extensive simulations would be necessary. However, these concepts should not be rejected out of hand, and the information gained through the associated simulations would increase our understanding of the magnetic fields and induced currents of any design.

Electronic Induction

Though the absolute power requirements will only be know once the TMS performance studies have been completed, to tell us the frequency and duration of stimulation, it is certain that we will be aiming at the absolute minimum power per pulse required for effective stimulation. This can be explored by 1) reducing resistive and inductive power loss in the “coil” and 2) altering pulse waveform for more efficient excitations, and resonant stimulator power. Resistive and inductive loss reduction will be sought through the uses of new “coil” materials, e.g., silver and/or room temperature superconductors, and “coil” conformation changes to confine losses to those associated with currents induced in the brain. We also explore the use of an impedance matching gel filled liner to take shape of head to see if this may improve current distribution or reduce power consumption. The shorter the stimulating waveform, the less power that is required to induce neuronal discharge, hence we design a stimulator power supply that puts out shorter waveforms, and, operates in a “resonant” mode to recapture the returning pulse.

Stimulation Control

According to exemplary embodiments, for stimulation control, the following are provided and modeled: a means for controlling the TMS stimulation, stimulation pattern control, and programmable lattice coil position control, which can be merely a means of activating the lattice elements to create a coil of a particular size at a particular position, or, it can include a test sequence for customizing the coil to accommodate individual anatomy.

Coil Designs, i.e., Possible Solution Devices

The following designs are modeled according to exemplary embodiments:
Standard figure-8;
Modified figure-8:
Programmable Lattice;
Vibrating magnets; and
MEMs—Micro-electromechanical Devices—Assess possible application in cortical stimulation phased array.

According to exemplary embodiments, several (at least 3) prototypes of the portable TMS system are produced and tested in conditions gradually approaching actual combat. The initial testing would be done in the BSL using military simulator computer programs. If these were successful, then testing would be performed in the actual field.

For each prototype delivered, the device is tested for performance capabilities (e.g., Tesla generated, heating, weight, etc.). Then, the prototype is used in the BSL on subjects and record performance behavior and other side effects.

Prototype 1 will be performance tested in the BSL, likely using flight simulators or submarine simulators, as well as the Sternberg task, by using likely simulators would be those involving long range flight simulators, or Advanced SEAL Delivery Systems.

Prototype 2 would be field tested in an armed services testing lab, and Prototype 3 would be tested in actual field conditions. Testing would be done under optimum and mildly sleep deprived conditions. Initial testing (prototype 1) would be done on healthy young men or women. Later testing would involve trained warfighters. Prototypes would be evaluated as well for safety (neuropsychological and behavioral as well as for the device itself).

In summary, the present invention provides, among other things, the following:
1) fMRI images: In one example, a series of fMRI activation maps in 120 healthy young men while performing the Sternberg task. These maps would show how much the functional localization of the Sternberg task varies across individuals.
2) TMS use parameter dose response performance data, both at baseline and after partial sleep deprivation; a detailed dose finding study of whether and how TMS might modify Sternberg performance if delivered during the task or before it. These TMS effects would be understood both at baseline and under conditions of partial sleep deprivation.
3) Interleaved TMS/fMRI maps showing how TMS applied at key regions modifies circuit behavior and changes performance.
4) Design maps and models of Man-portable TMS Systems: These would be detailed and able to be presented to industry for prototype construction.
5) 3 working prototypes of a man-portable TMS system.
6) Detailed data on whether and how these systems perform in simulator and field testing.
7) Improved understanding of how to create TMS systems for mass use without the need for individual fMRI guidance of TMS placement.
8) Understanding of whether TMS works at these parameters in women as well as men.

This body of work has the potential for revolutionizing the approach to enhancing cognitive performance by focusing on brain circuits and minimally invasive brain stimulation.

While there have been shown preferred and alternate embodiments of the present invention, it is to be understood that certain changes can be made in the form and arrangement of the elements of the system and steps of the method as would be know to one skilled in the art without departing from the underlying scope of the invention as described herein. Furthermore, the embodiments described above are only intended to illustrate the principles of the present invention and are not intended to limit the scope of the invention.

Moreover, the texts and drawings of the Appendix are incorporated into the application by reference as an integral part of the application. Additionally, the documents listed in the Appendix are incorporated into the application by reference.

1. A method for using transcranial magnetic stimulation to enhance cognitive performance, comprising the steps of:
locating at least one neural circuit in the brain of a subject, which is activated when the person performs a predetermined task;
positioning an electromagnetic coil over a region on the scalp of the subject corresponding to the at least one neural circuit in the brain of the subject; and
delivering a transcranial magnetic stimulation from the coil to the region on the scalp of the person to incude current to flow in the brain that causes neuronal depolarization in the brain and effectuates a change in the performance of the predetermined task by the subject.

2. The method of claim 1, wherein the subject is a human being.

3. A method for using transcranial magnetic stimulation to enhance cognitive performance in a plurality of subjects, comprising the steps of:
dividing the plurality of subjects into groups,
subjecting each of the groups into a first state and a second state,
locating at least one neural circuit in the brain of a subject in the group corresponding to one of the first state and the second state, which is activated when the subject performs a predetermined task under one of the first state and the second state,
positioning an electromagnetic coil over a region on the scalp of the subject corresponding to the at least one neural circuit in the brain of the subject, and
delivering a transcranial magnetic stimulation from the coil to the region on the scalp of the subject to induce a current to flow in the brain that causes neuronal depolarization in the brain and effectuates a change in the performance of the predetermined task by the subject under one of the first state and the second state.

4. The method of claim 3, wherein the subjects are human beings.

5. The method of claim 3, wherein the first state is a state in which a subject is at rest, and the second state is a state in which a subject is sleep-deprived.

6. The method of claim 3, wherein functional magnetic resonance imaging maps are used to identify different neural circuits associated with different subject on a state, wherein the neural circuits are activated while a predetermined task is performed.

7. The method of claim 6, wherein transcranial magnetic stimulation is delivered to proper neutral circuits to restore and/or retrain the circuits to enhance the performance.

8. A method of using transcranial magnetic stimulation to enhance cognitive performance in at least one subject, comprising:
during a behavior individualized imaging of at least one cognitive neural circuit, locating the at least one cognitive neural circuit, individually positioning an electromagnetic coil over a region on the scalp of the subject corresponding to the at least one cognitive neural circuit, and delivering a stimulation through the electromagnetic coil to the at least one cognitive neural circuit to affect the behavior related to the at least one cognitive neural circuit.

9. The method of claim 8, wherein individualized imaging can be performed by a functional magnetic resonance imaging scanner.

10. The method of claim 8, wherein the electromagnetic coil is interleaved with a transcranial magnetic stimulation system to provide synergistic stimulation(s).

11. A system for using transcranial magnetic stimulation to enhance cognitive performance in at least one subject, comprising:
means for locating at least one neural circuit in the brain of a subject, which is activated when the subject performs a predetermined task,
an electromagnetic coil that can be positioned over a spot on the scalp of the subject corresponding to the at least one neural circuit in the brain of the subject, and
means for delivering a transcranial magnetic stimulation from the coil to the spot on the scalp of the subject so as to induce a current to flow in the brain, cause neuronal depolarization in the brain, and effectuate a change in the performance of the predetermined task by the subject.

12. The system of claim 11, wherein the locating means includes a functional magnetic resonance imaging system that can be utilized to scan and generate maps of the interested neural circuits so as to locate proper neural circuits responsible for a predetermined task.

13. The system of claim 11, further comprising a computer having a CPU and one or more memory devices to coordinate the operation among the different parts of the system, optimize the operation parameters, and facilitate the transcranial magnetic stimulation delivering.

14. The system of claim 13, wherein the operation parameters are transcranial magnetic stimulation parameters.

15. A portable system for using transcranial magnetic stimulation to enhance cognitive performance in at least one subject, the system comprising:
a CPU;
an energy source electrically coupled to the CPU;
a database in communication with the CPU and having functional magnetic resonance imaging (fMRI) maps of neural circuits corresponding to a plurality of tasks stored therein; and
a movable electromagnetic coil electrically coupled to the energy source and in communication with the CPU, wherein when a subject is to perform a predetermined task, the CPU selects one or more fMRI maps of one or more neural circuits from the corresponding to the predetermined task from the database and causes the movable electromagnetic coil to be positioned over a region on the scalp of the subject according to the selected one or more fMRI maps, and the movable electromagnetic coil delivers transcranial magnetic stimulation to the region on the scalp of the subject so as to induce a current to flow in the brain, cause neuronal depolarization in the brain, and effectuate a change in the performance of the predetermined task by the subject.

16. The system of claim 15, wherein the energy source is a battery.

17. The system of claim 15, wherein the database is associated with a memory device of the CPU and/or a separate memory device.

18. The system of claim 15, wherein the subject is a person.

19. The system of claim 15, wherein the subject is an animal.

20. The system of claim 15, wherein the system is constructed within a frame that is portable.

21. The system of claim 15, wherein the system comprises an array of transcranial magnetic stimulation coils, each being able to deliver transcranial magnetic stimulation individually or in coordination.
